(19) United States
(12) Patent Application Publication
(10) Pub. No.: US 2014/0256800 A1
(43) Pub. Date: Sep. 11, 2014

(54) FEV MINI-PROMOTERS

(71) Applicant: The University of British Columbia, Vancouver (CA)

(72) Inventors: Elizabeth M. Simpson, Vancouver (CA); Wyeth W. Wasserman, Vancouver (CA); Daniel Goldowitz, Port Moody (CA); Robert A. Holt, Vancouver (CA); Steven J. Jones, Vancouver (CA); Charles de Leeuw, Vancouver (CA); Elodie Portales-Casamar, Vancouver (CA); Cletus D'Souza, Vancouver (CA); Vikramjit Chopra, Vancouver (CA)

(21) Appl. No.: 14/195,290

(22) Filed: Mar. 3, 2014

Related U.S. Application Data
(60) Provisional application No. 61/773,673, filed on Mar. 6, 2013.

(51) Int. Cl. A61K 48/00; C12N 15/85; C07K 14/005

(52) U.S. Cl. CPC A61K 48/0058; C07K 14/005; C12N 15/85
USPC 514/44 R; 536/24.1; 435/320.1; 435/325; 435/455

(57) ABSTRACT

Isolated polynucleotides comprising a FEV mini-promoters are provided. The mini-promoter may be operably linked to an expressible sequence, e.g. reporter genes, genes encoding a polypeptide of interest, regulatory RNA sequences such as miRNA, siRNA, anti-sense RNA, etc., and the like. In some embodiments a cell comprising a stable integrant of an expression vector is provided, which may be integrated in the genome of the cell. The promoter may also be provided in a vector, for example in combination with an expressible sequence. The polynucleotides find use in a method of expressing a sequence of interest, e.g. for identifying or labeling cells, monitoring or tracking the expression of cells, gene therapy, etc.

### FIELD OF THE INVENTION

The invention relates to gene promoters and regulatory elements. More specifically, the invention relates to novel FEV promoter compositions and related methods.

### BACKGROUND

There is a need for characterized human FEV promoters for gene expression, for instance in human gene therapy applications. It is in particular useful to identify small promoter elements that are sufficient to drive expression in certain cell types, for instance retinal ganglion cells or retinal amacrine cells. Such small promoter elements, or “mini-promoters” are particularly useful in certain applications, for instance they are more amenable to insertion into viral vectors used in gene therapy applications.

Endogenous expression patterns for FEV are described in the art:
Hendricks T, Francis N, Fyodorov D, Deneris E S. 1999. The ETS domain factor Pet-1 is an early and precise marker of central serotonin neurons and interacts with a conserved element in serotonergic genes. J Neurosci 19: 10348-10356.
Hendricks T J, Fyodorov D V, Wegman L J, Lelutiu N B, Pehek E A, Yamamoto B, Silver J, Weeber E J, Sweatt J D, Deneris E S. 2003. Pet-1 ETS gene plays a critical role in 5-HT neuron development and is required for normal anxiety-like and aggressive behavior. Neuron 37: 233-247.
Iyo A H, Porter B, Deneris E S, Austin M C. 2005. Regional distribution and cellular localization of the ETS-domain transcription factor, FEV, mRNA in the human postmortem brain. Synapse 57: 223-228.
Maurer P, Rorive S, de Kerchove d'Exaerde A, Schiffmann S N, Salmon I, de Launoit Y. 2004. The Ets transcription factor Fev is specifically expressed in the human central serotonergic neurons. Neuroscience Letters 357: 215-218.

### SUMMARY OF THE INVENTION

The present invention provides novel nucleic acid sequence compositions and methods relating to minimal human FEV promoters. The invention is based in part on the surprising discovery that certain minimal FEV promoter elements are capable of expressing in specific cell types, for instance in cells of the brain or eye.

In one embodiment of the invention, there is provided an isolated nucleic acid fragment comprising a FEV mini-promoter, wherein the FEV mini-promoter comprises a FEV basal promoter. The FEV mini-promoter may have a nucleic acid sequence which is substantially similar in sequence and function to SEQ ID NO: 1 or 2. The FEV mini-promoters may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule.

In one embodiment, there is provided an expression vector comprising a FEV mini-promoter, wherein the FEV mini-promoter comprises a FEV basal promoter. The FEV basal promoter may have a nucleic acid sequence which is substantially similar in sequence and function to SEQ ID NO: 1 or 2. The FEV mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may further comprise a genomic targeting sequence. The genomic targeting sequence may be HPRT.

In one embodiment, there is provided a method for expressing a gene, protein, RNA interference molecule or the like in a cell, the method comprising introducing into the cell an expression vector comprising a FEV mini-promoter element, wherein the FEV mini-promoter element comprises a FEV basal promoter element. Cells of interest include, without limitation, cells of the peripheral or central nervous system and progenitors thereof, e.g. embryonic stem cells, neural stem cells, neurons, glial cells, astrocytes, microgial cells, etc; and/or cells in the eye and progenitors thereof, e.g. retinal cells, amacrine cells, etc. The FEV mini-promoter may have a nucleic acid sequence which is substantially similar in sequence and function to SEQ ID NO: 1 or 2. The FEV mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may thus further comprise a genomic targeting sequence. The genomic targeting sequence may be HPRT.

In one embodiment of the invention, there is provided a method for identifying or labeling a cell, the method comprising introducing into the cell an expression vector comprising a FEV mini-promoter element, wherein the FEV mini-promoter element comprises a FEV basal promoter element, and wherein the expressible sequence comprises a reporter gene. The FEV mini-promoter element may have a nucleic acid sequence substantially similar in sequence and function to SEQ ID NO: 1 or 2. In some embodiments, the cell is a peripheral or central nervous system cell or progenitors thereof, including, without limitation, embryonic stem cells, neural stem cells, glial cells, astrocytes, neurons and the like, and/or cells in the eye and progenitors thereof, e.g. retinal cells, retinal ganglion cells, retinal amacrine cells, etc. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, RNA interference molecule and the like.

In one embodiment of the invention, there is provided a method for monitoring or tracking the development or maturation of a cell, the method comprising: 1) introducing into the cell a expression vector comprising a FEV mini-promoter element operably linked to an expressible sequence, wherein the FEV mini-promoter element comprises a FEV basal promoter element, and wherein the expressible sequence comprises a reporter gene; and 2) detecting the expression of the reporter gene in the cell of in progeny of the cell as a means of determining the lineage, identity or developmental state of the cell or cell progeny. The FEV mini-promoter element may have a nucleic acid sequence substantially similar in sequence and function to SEQ ID NO: 1 or 2. In some embodiments, the cell is a peripheral or central nervous system cell or progenitors thereof, including, without limitation, embryonic stem cells, neural stem cells, glial cells, neurons and the like. In some embodiments, the cell is an eye cell or progenitor thereof, including without limitation a retinal cell, a retinal ganglion cell, retinal amacrine cell, and the like.

In certain embodiments of the invention, there is thus provided a method of treatment of a subject having a disease or condition of the eye, the method comprising administering to the subject a therapeutically effective dose of a composition comprising a FEV mini-promoter element, wherein the FEV mini-promoter element comprises a FEV basal promoter element. The FEV mini-promoter element may have a nucleic acid sequence substantially similar in sequence and function to SEQ ID NO: 1 or 2. The disease or condition may be chosen from: retinal diseases, retinal degeneration, retinal damage, blindness, macular degeneration, retinitis pigmentosa, inherited retinal genetic diseases, diabetic retinopathy, cone rod dystrophy, hypertensive/diabetic retinopathy. The therapeutic or beneficial compound may be a light-sensitive compound, for instance rhodopsin, channel rhodopsin, etc.

In certain embodiments of the invention, there is thus provided a method of treatment of a subject having a disease or condition of the central nervous system, the method comprising administering to the subject a therapeutically effective dose of a composition comprising a FEV mini-promoter element, wherein the FEV mini-promoter element comprises a FEV basal promoter element. The FEV mini-promoter element may have a nucleic acid sequence substantially similar in sequence and function to SEQ ID NO: 1 or 2. The disease or condition may be related to or associated with serotonergic cells in the brain. The disease or condition may be chosen from: depression, bipolar disorder, anxiety, Parkinson's Disease, Huntington's Disease, Alzheimer's Disease, cognitive disorders, dementia, mood disorders, and schizophrenia.

### SHORT DESCRIPTION OF FIGURES

FIG. 1—Ple67-lacZ (FEV) expression in the brain. Mice were perfused, brains harvested and stained with X-gal. Expression of β-galactosidase from the MiniPromoter as detected by X-gal substrate is shown in blue. Ple67-lacZ expresses in known serotonergic mouse brain regions. (A) 1-mm sagittal section of mouse brain stained with X-gal substrate (blue indicates lacZ enzyme activity). (B) 1-mm coronal mouse brain section. (A, B) The Raphe Nuclei of the midbrain reticular formation, pontine reticular formation and the medulla oblongata are indicated with white arrows.

FIG. 2—Ple53-lacZ (FEV) expression in the eye. Eyes were perfused and stained with X-gal to detect β-galactosidase expression from the MiniPromoter. Ple67 expression in ganglion cell layer (GCL) of the retina. Eyes were stained for lacZ activity using the X-gal substrate (blue) and counter-stained for nuclei with neutral red. The ganglion cell layer (GCL) is shown at the top and outer nuclear layer at the bottom (ONL). Positive cells are found occasionally in the GCL, and rarely in the innermost part of the INL (not shown). Staining is indicative of amacrine and/or retinal ganglion cells.

FIG. 3—DNA expression vector (pEMS1313) into which FEV promoter elements were inserted for expression studies. The FEV promoter with a nucleic acid sequence corresponding to SEQ ID NO: 1 or 2 was inserted into the multiple cloning site (MCS) of the pEMS1313 vector such that it became operably linked to the lac Z reporter gene. The final construct, called Ple67 (containing SEQ ID NO: 1) or Ple313 (SEQ ID NO: 2) also contained the HPRT genomic targeting sequence, an ampicillin resistance gene (AmpR) for screening, and a transcriptional termination sequence (SV40 polyA), as well as other elements necessary for vector replication and gene expression.

### DETAILED DESCRIPTION

The compositions of the present invention include novel polynucleotides comprising FEV promoter elements (also referred to herein as FEV mini-promoters) as well as novel expression vectors comprising said FEV promoter elements (or mini-promoters). The present invention also includes various methods utilizing these novel FEV promoter (or mini-promoter) elements or expression vectors.

The term ‘FEV’ refers to the gene which encodes the FEV protein. The human homolog of FEV is encoded by the human gene identified as EntrezGene #54738. The protein encoded by human FEV has the Protein Accession #Q99581.1, however other protein accession numbers may also be assigned to this protein. FEV may also include other isoforms and/or splice variants. Other mammalian FEV homologs may include but are not limited to: Rattus norvegicus (EntrezGene #246271), Mus musculus (EntrezGene #260298).

The term ‘promoter’ refers to the regulatory DNA region which controls transcription or expression of a gene and which can be located adjacent to or overlapping a nucleotide or region of nucleotides at which RNA transcription is initiated. A promoter contains specific DNA sequences which bind protein factors, often referred to as transcription factors, which facilitate binding of RNA polymerase to the DNA leading to gene transcription. A ‘basal promoter’, also referred to as a ‘core promoter’, usually means a promoter which contains all the basic necessary elements to promote transcriptional expression of an operably linked polynucleotide. Eukaryotic basal promoters typically, though not necessarily, contain a TATA-box and/or a CAAT box. A ‘FEV basal promoter’, in the context of the present invention and as used herein, is a nucleic acid compound having a sequence with at least 65%, at least 70%, at least 80%, at least 85%, at least 90%, at least 95%, or at least 99% similarity to SEQ ID NO: 1 or 2. The basal promoter may be truncated relative to SEQ ID NOs: 1 and 2, e.g. by a deletion of about 10, 20, 30 40, 50, 75, 100, or more nucleotides at either terminus.

The term ‘operably linked’, in the context of the present invention, means joined in such a fashion as to work together to allow transcription. In some embodiments of the invention, two polynucleotide sequences may be operably linked by being directly linked via a nucleotide bond. In this fashion, the two operably linked elements contain no intervening sequences and in being joined are able to direct transcription of an expression sequence. In other embodiments of the invention, two elements may be operably linked by an intervening compound, for instance a polynucleotide sequence of variable length. In such a fashion, the operably linked elements, although not directly juxtaposed, are still able to direct transcription of an expression sequence. Thus, according to some embodiments of the invention, one or more promoter elements may be operably linked to each other, and additionally be operably linked to a downstream expression sequence, such that the linked promoter elements are able to direct expression of the downstream expression sequence.

The term ‘mini-promoter’ refers to a promoter in which certain promoter elements are selected from an endogenous full length promoter for a gene, usually in such a fashion as to reduce the overall size of the promoter compared to the native sequence. For example, after identification of critical promoter elements, using one or more of various techniques, the native sequences that intervene between identified elements may be partially or completely removed. Other non-native sequences may optionally be inserted between the identified promoter elements. Promoter sequences such as enhancer elements may have an orientation that is different from the native orientation—for example, a promoter element may be inverted, or reversed, from its native orientation. Alternatively, selecting a minimal basal promoter that is sufficient to drive expression in particular cells or tissues may also be desirable. Since promoter elements that impact expression patterns are known to be distributed over varying distances of the proximal and/or distal endogenous promoter, it is a non-trivial task to identify a mini-promoter comprising a minimal basal promoter and optional regulatory regions that will adequately express in the desired cell or tissue types. A mini-promoter may provide certain advantages over native promoter conformations. For example, the smaller size of the mini-promoter may allow easier genetic manipulation, for example in the design and/or construction of expression vectors or other recombinant DNA constructs. In addition, the smaller size may allow easier insertion of DNA constructs into host cells and/or genomes, for example via transfection, transformation, etc. Other advantages of mini-promoters are apparent to one of skill in the art. In some embodiments of the invention, there are thus provided novel FEV mini-promoters comprising a FEV basal promoter. The FEV mini-promoters drive expression in various novel cell and tissue types, and thus may have novel and useful applications.

The term ‘expressible sequence’ refers to a polynucleotide composition which is operably linked to a promoter element such that the promoter element is able to cause transcriptional expression of the expression sequence. An expressible sequence is typically linked downstream, on the 3′-end of the promoter element(s) in order to achieve transcriptional expression. The result of this transcriptional expression is the production of an RNA macromolecule. The expressed RNA molecule may encode a protein and may thus be subsequently translated by the appropriate cellular machinery to produce a polypeptide protein molecule. In some embodiments of the invention, the expression sequence may encode a reporter protein. Alternately, the RNA molecule may be an antisense, RNAi or other non-coding RNA molecule, which may be capable of modulating the expression of specific genes in a cell, as is known in the art.

The term ‘RNA’ as used in the present invention includes full-length RNA molecules, which may be coding or non-coding sequences, fragments, and derivatives thereof. For example, a full-length RNA may initially encompass up to about 20 Kb or more of sequence, and frequently will be processed by splicing to generate a small mature RNA. Fragments, RNAi, miRNA and anti-sense molecules may be smaller, usually at least about 18 nt. in length, at least about 20 nt in length, at least about 25 nt. in length, and may be up to about 50 nt. in length, up to about 100 nt in length, or more. RNA may be single stranded, double stranded, synthetic, isolated, partially isolated, essentially pure or recombinant. RNA compounds may be naturally occurring, or they may be altered such that they differ from naturally occurring RNA compounds.

Alterations may include addition, deletion, substitution or modification of existing nucleotides. Such nucleotides may be either naturally occurring, or non-naturally occurring nucleotides. Alterations may also involve addition or insertion of non-nucleotide material, for instance at the end or ends of an existing RNA compound, or at a site that is internal to the RNA (ie. between two or more nucleotides).

The term ‘nucleic acid’ as used herein includes any nucleic acid, and may be a deoxyribonucleotide or ribonucleotide polymer in either single or double-stranded form. A ‘polynucleotide’ or ‘nucleotide polymer’ as used herein may include synthetic or mixed polymers of nucleic acids, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e. g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

A ‘purine’ is a heterocyclic organic compound containing fused pyrimidine and imidazole rings, and acts as the parent compound for purine bases, adenine (A) and guanine (G). ‘Nucleotides’ are generally a purine (R) or pyrimidine (Y) base covalently linked to a pentose, usually ribose or deoxyribose, where the sugar carries one or more phosphate groups. Nucleic acids are generally a polymer of nucleotides joined by 3′ 5′ phosphodiester linkages. As used herein ‘purine’ is used to refer to the purine bases, A and G, and more broadly to include the nucleotide monomers, deoxyadenosine-5′-phosphate and deoxyguanosine-5′-phosphate, as components of a polynucleotide chain. A ‘pyrimidine’ is a single-ringed, organic base that forms nucleotide bases, such as cytosine (C), thymine (T) and uracil (U). As used herein ‘pyrimidine’ is used to refer to the pyrimidine bases, C, T and U, and more broadly to include the pyrimidine nucleotide monomers that along with purine nucleotides are the components of a polynucleotide chain.

It is within the capability of one of skill in the art to modify the sequence of a promoter nucleic acid sequence, e.g. the provided basal promoter and regulatory sequences, in a manner that does not substantially change the activity of the promoter element, i.e. the transcription rate of an expressible sequence operably linked to a modified promoter sequence is at least about 65% the transcription rate of the original promoter, at least about 75% the transcription rate of the original promoter sequence, at least about 80%, at least about 90%, at least about 95%, at least about 99%, or more. Such modified sequences would be considered to be ‘functionally similar’ or to have ‘functional similarity’ or ‘substantial functional similarity’ to the unmodified sequence. Such modifications may include insertions, deletions which may be truncation of the sequence or internal deletions, or substitutions. The level of sequence modification to an original sequence will determine the ‘sequence similarity’ of the original and modified sequences. Modification of the promoter elements of the present invention in a fashion that does not significantly alter transcriptional activity, as described above would result in sequences with ‘substantial sequence similarity’ to the original sequence i.e. the modified sequence has a nucleic acid composition that is at least about 65% similar to the original promoter sequence, at least about 75% similar to the original promoter sequence, at least about 80%, at least about 90%, at least about 95%, at least about 99%, or more similar to the original promoter sequence. Thus, mini-promoter elements which have substantial functional and/or sequence similarity are herein described and are within the scope of the invention.

An ‘RNA interference molecule’, or ‘RNA interference sequence’ as defined herein, may include, but is not limited to, an antisense RNA molecule, a microRNA molecule or a short hairpin RNA (shRNA) molecule. Typically, RNA interference molecules are capable of target-specific modulation of gene expression and exert their effect either by mediating degradation of the mRNA products of the target gene, or by preventing protein translation from the mRNA of the target gene. The overall effect of interference with mRNA function is modulation of expression of the product of a target gene. This modulation can be measured in ways which are routine in the art, for example by Northern blot assay or reverse transcriptase PCR of mRNA expression, Western blot or ELISA assay of protein expression, immunoprecipitation assay of protein expression, etc.

An ‘antisense RNA molecule’, as used herein, is typically a single stranded RNA compound which binds to complementary RNA compounds, such as target mRNA molecules, and blocks translation from the complementary RNA compounds by sterically interfering with the normal translational machinery. Specific targeting of antisense RNA compounds to inhibit the expression of a desired gene may design the antisense RNA compound to have a homologous, complementary sequence to the desired gene. Perfect homology is not necessary for inhibition of expression. Design of gene specific antisense RNA compounds, including nucleotide sequence selection and additionally appropriate alterations, are known to one of skill in the art.

The term ‘microRNA molecule’, ‘microRNA’ or ‘miRNA’, as used herein, refers to single-stranded RNA molecules, typically of about 21-23 nucleotides in length, which are capable of modulating gene expression. Mature miRNA molecules are partially complementary to one or more messenger RNA (mRNA) molecules, and their main function is to downregulate gene expression. Without being bound by theory, miRNAs are first transcribed as primary transcripts or pri-miRNA with a cap and poly-A tail and processed to short, 70-nucleotide stem-loop structures known as pre-miRNA in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules and induce mRNA degradation by argonaute proteins, the catalytically active members of the RISC complex. Animal miRNAs are usually complementary to a site in the 3′ UTR whereas plant miRNAs are usually complementary to coding regions of mRNAs.

The term ‘short hairpin RNA’ or ‘shRNA’ refers to RNA molecules having an RNA sequence that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA Polymerase III whereas miRNA is transcribed by RNA Polymerase II. Techniques for designing target specific shRNA molecules are known in the art.

An ‘expression vector’ is typically a nucleic acid molecule which may be integrating or autonomous, (i.e. self-replicating), and which contains the necessary components to achieve transcription of an expressible sequence in a target cell, when introduced into the target cell. Expression vectors may include plasmids, cosmids, phage, YAC, BAC, mini-chromosomes, viruses, e.g. retroviruses, adenovirus, lentivirus, SV-40, and the like; etc. Many such vectors have been described in the art and are suitable for use with the promoters of the present invention. Expression vectors of the present invention include a promoter as described herein, operably linked to an expressible sequence, which may also be optionally operably linked to a transcription termination sequence, such as a polyadenylation sequence. The expression vector optionally contains nucleic acid elements which confer host selectivity, elements that facilitate replication of the vector, elements that facilitate integration of the vector into the genome of the target cell, elements which confer properties, for example antibiotic resistance, to the target cell which allow selection or screening of transformed cells and the like. Techniques and methods for design and construction of expression vectors are well known in the art.

It may be desirable, when driving expression of an expressible sequence with a particular promoter system to have the expression occur in a stable and consistent manner. A factor that has been shown to affect expression is the site of integration of an expression vector or construct into the genome of the target cell, sometimes called ‘position effects’. Such position effects may be caused by, for example, local chromatin structure which affects expression of sequences from that region of the genome. One method to control for position effects when integrating an expression vector or construct into the genome of a target cell is to include a ‘genomic targeting sequence’ in the vector or construct that directs integration of the vector or construct to a specific genomic site. As an example, the hypoxanthine phosphoribosyltransferase (HPRT) gene has been used successfully for this purpose (Bronson, Plaehn et al. 1996; Jasin, Moynahan et al. 1996). The HPRT gene has additional advantages as a genomic targeting sequence, for instance its concomitant use as a selectable marker system. Other genomic targeting sequences that may be useful in the present invention are described in the art, for instance (Jasin, Moynahan et al. 1996; van der Weyden, Adams et al. 2002). The genomic targeting signals as described herein are useful in certain embodiments of the present invention.

Introduction of nucleic acids or expression vectors into cells may be accomplished using techniques well known in the art, for example microinjection, electroporation, particle bombardment, or chemical transformation, such as calcium-mediated transformation, as described for example in Maniatis et al. 1982, Molecular Cloning, A laboratory Manual, Cold Spring Harbor Laboratory or in Ausubel et al. 1994, Current protocols in molecular biology, JoIm Wiley and Sons.

In certain embodiments of the invention, there are provided methods of treatment using the nucleic acids or expression vectors, for instance for gene therapy applications. The nucleic acids or expression vectors of the present invention may be administered in isolation, or may be linked to or in combination with tracer compounds, liposomes, carbohydrate carriers, polymeric carriers or other agents or excipients as will be apparent to one of skill in the art. In an alternate embodiment, such compounds may comprise a medicament, wherein such compounds may be present in a pharmacologically effective amount.

The term ‘medicament’ as used herein refers to a composition that may be administered to a patient or test subject and is capable of producing an effect in the patient or test subject. The effect may be chemical, biological or physical, and the patient or test subject may be human, or a non-human animal, such as a rodent or transgenic mouse, or a dog, cat, cow, sheep, horse, hamster, guinea pig, rabbit or pig. The medicament may be comprised of the effective chemical entity alone or in combination with a pharmaceutically acceptable excipient.

The term ‘pharmaceutically acceptable excipient’ may include any and all solvents, dispersion media, coatings, antibacterial, antimicrobial or antifungal agents, isotonic and absorption delaying agents, and the like that are physiologically compatible. An excipient may be suitable for intravenous, intraperitoneal, intramuscular, subcutaneous, intrathecal, intraocular, topical or oral administration. An excipient may include sterile aqueous solutions or dispersions for extemporaneous preparation of sterile injectable solutions or dispersion. Use of such media for preparation of medicaments is known in the art.

The nucleic acids or expression vectors of the present invention may be administered to a subject using a viral delivery system. For instance, the nucleic acids may be inserted into a viral vector using well known recombinant techniques. The subsequent viral vector may then be packaged into a virus, such as adenovirus, lentivirus, attenuated virus, adeno-associated virus (AAV), and the like. Viral delivery for gene therapy applications is well known in the art. There exist a variety of options for viruses suitable for such delivery, which may also involve selecting an appropriate viral serotype for delivery and expression in an appropriate tissue.

Compositions or compounds according to some embodiments of the invention may be administered in any of a variety of known routes. Examples of methods that may be suitable for the administration of a compound include orally, intravenous, inhalation, intramuscular, subcutaneous, topical, intraperitoneal, intra-ocular, intra-rectal or intra-vaginal suppository, sublingual, and the like. The compounds of the present invention may be administered as a sterile aqueous solution, or may be administered in a fat-soluble excipient, or in another solution, suspension, patch, tablet or paste format as is appropriate. A composition comprising the compounds of the invention may be formulated for administration by inhalation. For instance, a compound may be combined with an excipient to allow dispersion in an aerosol. Examples of inhalation formulations will be known to those skilled in the art. Other agents may be included in combination with the compounds of the present invention to aid uptake or metabolism, or delay dispersion within the host, such as in a controlled-release formulation. Examples of controlled release formulations will be known to those of skill in the art, and may include microencapsulation, embolism within a carbohydrate or polymer matrix, and the like. Other methods known in the art for making formulations are found in, for example, “Remington's Pharmaceutical Sciences”, (19th edition), ed. A. Gennaro, 1995, Mack Publishing Company, Easton, Pa.

The dosage of the compositions or compounds of some embodiments of the invention may vary depending on the route of administration (oral, intravenous, inhalation, or the like) and the form in which the composition or compound is administered (solution, controlled release or the like). Determination of appropriate dosages is within the ability of one of skill in the art. As used herein, an ‘effective amount’, a ‘therapeutically effective amount’, or a ‘pharmacologically effective amount’ of a medicament refers to an amount of a medicament present in such a concentration to result in a therapeutic level of drug delivered over the term that the drug is used. This may be dependent on mode of delivery, time period of the dosage, age, weight, general health, sex and diet of the subject receiving the medicament. Methods of determining effective amounts are known in the art. It is understood that it could be potentially beneficial to restrict delivery of the compounds of the invention to the target tissue or cell in which protein expression. It is also understood that it may be desirable to target the compounds of the invention to a desired tissue or cell type. The compounds of the invention may thus be coupled to a targeting moiety. The compounds of the invention may be coupled to a cell uptake moiety. The targeting moiety may also function as the cell uptake moiety.

FEV Mini-Promoters

The present invention herein provides FEV mini-promoter sequences which are capable of effecting novel transcriptional expression in a spatial and temporal fashion in the brain and/or eye. Also provided are expression vector compositions comprising FEV mini-promoters which allow consistent specific spatiotemporal transcription of expression sequences. Also provided are novel methods utilizing these FEV mini-promoters and expression vectors. The FEV promoters of the invention, as described herein, are referred to as ‘mini-promoters’ to reflect the fact that the mini-promoters comprise minimal FEV promoter elements sufficient to drive expression.

The inventors have demonstrated, as illustrated in the non-limiting Working Examples, that human FEV mini-promoters having a sequence corresponding to SEQ ID NO: 1 and 2 (also referred to in the Working Examples as Ple67 and Ple313), and which is comprised a human FEV basal promoter, is capable of directing expression of an expressible sequence which is operably linked downstream of the FEV promoter in specific cell types in different regions of the brain and/or eye. It is within the skill of one in the art to locate and determine these relative positions based on published sequence information for this gene, for instance found in the GenBank or PubMed public databases. It is understood that these genomic coordinates and relative positions are provided for the purposes of context, and that if any discrepancies exist between published sequences and the sequence listings provided herein, then the sequence listings shall prevail.

Promoters of the present invention may be modified with respect to the native basal promoter sequence. In general, such modifications will not change the functional activity of the promoter with respect to cell-type selectivity; and to the rate of transcription in cells where the promoter is active. The modified promoter provide for a transcription rate of an expressible sequence operably linked to a modified promoter sequence that is at least about 75% the transcription rate of the promoter sequence of SEQ ID NO: 1 or 2, at least about 80%, at least about 90%, at least about 95%, at least about 99%, or more. Methods of assessing promoter strength and selectivity are known in the art, including, for example, expression of a reporter sequence in a cell in vivo or in vitro, and quantitating the reporter activity.

Modifications of Sequences set forth in SEQ ID NO: 1 or 2 that are not conserved may be deleted or substituted, usually modifications that retain the spacing between conserved sequences is preferred

In one embodiment of the invention, there is provided an isolated nucleic acid fragment comprising a FEV mini-promoter, wherein the FEV mini-promoter comprises a FEV basal promoter. The FEV mini-promoter may have a nucleic acid sequence which is substantially similar in sequence and function to SEQ ID NO: 1 or 2. The FEV mini-promoters may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule.

It is an object of the present invention to provide means of expressing a gene, protein, RNA interference molecule or the like in a cell, tissue or organ. As such, the inventors thus provide novel expression vectors comprising FEV mini-promoters which are capable of accomplishing this task. In one embodiment, there is provided an expression vector comprising a FEV mini-promoter, wherein the FEV mini-promoter comprises a FEV basal promoter. The FEV mini-promoter may have a nucleic acid sequence which is substantially similar in sequence and function to SEQ ID NO: 1 or 2. The FEV mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may further comprise a genomic targeting sequence. The genomic targeting sequence may be HPRT, e.g. human HPRT, mouse HPRT, etc.

The inventors have herein demonstrated that expression vectors comprising novel FEV mini-promoter elements are capable of directing transcription of an expression sequence in specific cell types, for instance in ganglion or amacrine cells in the retina (eye) or in serotonergic cells in the brain. In one embodiment of the invention, there is thus provided a method for expressing a gene, protein, RNA interference molecule or the like in a cell, the method comprising introducing into the cell an expression vector comprising a FEV mini-promoter element, wherein the FEV mini-promoter element comprises a FEV basal promoter element. Cells of interest include, without limitation, cells of the peripheral or central nervous system and progenitors thereof, e.g. embryonic stem cells, neural stem cells, neurons, glial cells, astrocytes, microglial cells, etc; and/or cells in the eye and progenitors thereof, e.g. retinal cells, retinal ganglion cells, retinal amacrine cells etc. The FEV mini-promoter may have a nucleic acid sequence which is substantially similar in sequence and function to SEQ ID NO: 1 or 2. The FEV mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, and the like. The expressible sequence may encode an RNA interference molecule. The expression vector may thus further comprise a genomic targeting sequence. The genomic targeting sequence may be HPRT.

In one embodiment of the invention, there is provided a method for identifying or labeling a cell, the method comprising introducing into the cell an expression vector comprising a FEV mini-promoter element, wherein the FEV mini-promoter element comprises a FEV basal promoter element, and wherein the expressible sequence comprises a reporter gene. In other embodiments, the FEV mini-promoter comprises a FEV basal promoter. The FEV mini-promoter element may have a nucleic acid sequence substantially similar in sequence and function to SEQ ID NO: 1 or 2. The inventors have demonstrated that expression vectors comprising certain human FEV promoter elements are capable of expression in specific regions of the brain and eye, most notably serotonergic cells in the brain and retinal ganglion cells and amacrine cells in the eye. In some embodiments, the cell is a peripheral or central nervous system cell or progenitors thereof, including, without limitation, embryonic stem cells, neural stem cells, glial cell, neuronal cells, astrocytes, and the like. In some embodiments, the cell is a cell of the eye and progenitors thereof, including without limitation retinal cells, retinal ganglion cells, retinal amacrine cells, and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, RNA interference molecule and the like.

In further embodiments of the invention, there is provided a method for monitoring or tracking the development or maturation of a cell, the method comprising: 1) introducing into the cell a expression vector comprising a FEV mini-promoter element operably linked to an expressible sequence, wherein the FEV mini-promoter element comprises a FEV basal promoter element, and wherein the expressible sequence comprises a reporter gene; and 2) detecting the expression of the reporter gene in the cell of in progeny of the cell as a means of determining the lineage, identity or developmental state of the cell or cell progeny. The FEV mini-promoter element may have a nucleic acid sequence substantially similar in sequence and function to SEQ ID NO: 1 or 2. In such a fashion, one may be able to follow the development of a parent cell as it differentiates into more mature cells. As an example, one could introduce an expression vector comprising the aforementioned FEV mini-promoter elements into a pluripotent stem cell, monitor the expression of the reporter gene that is being expressed by the FEV promoter elements during the maturation and differentiation of the stem cell and thus determine the state of maturation, for instance in the differentiation of the pluripotent stem cell into a specific brain or retinal cell type. The inventors have demonstrated that the FEV mini-promoter elements described herein direct transcriptional expression in certain brain and retinal cell types, and so detection of reporter gene expression in a cell would thus be indicative of the cellular identity of the cell as being a certain type of brain or retinal cell.

The inventors have herein demonstrated that certain FEV mini-promoter elements of the present invention are capable of driving expression in serotonergic brain cells, and in retinal ganglion and retinal amacrine cells. This surprising expression pattern provides additional methods of use for these mini-promoter elements. For instance, it may be desirable to utilize the FEV mini-promoters of the present invention in a gene therapy or cell therapy application wherein the FEV mini-promoters are utilized to drive expression of a therapeutic or beneficial compound, such as a protein, in serotonergic brain cells or retinal ganglion or retinal amacrine cells. In such a way, the therapeutic or beneficial compound may be useful for a disease or condition that involves such cells, or which may be improved by expression of the therapeutic or beneficial compound in those cells. In certain embodiments of the invention, there is thus provided a method of treatment of a subject having a disease or condition of the eye, the method comprising administering to the subject a therapeutically effective dose of a composition comprising a FEV mini-promoter element, wherein the FEV mini-promoter element comprises a FEV basal promoter element. The FEV mini-promoter element may have a nucleic acid sequence substantially similar in sequence and function to SEQ ID NO: 1 or 2. The disease or condition may be chosen from: retinal diseases, retinal degeneration, retinal damage, blindness, macular degeneration, retinitis pigmentosa, inherited retinal genetic diseases, diabetic retinopathy, cone rod dystrophy, hypertensive/diabetic retinopathy. The therapeutic or beneficial compound may be a light-sensitive compound, for instance rhodopsin, channel rhodopsin, etc.

In certain embodiments of the invention, there is thus provided a method of treatment of a subject having a disease or condition of the central nervous system, the method comprising administering to the subject a therapeutically effective dose of a composition comprising a FEV mini-promoter element, wherein the FEV mini-promoter element comprises a FEV basal promoter element. The FEV mini-promoter element may have a nucleic acid sequence substantially similar in sequence and function to SEQ ID NO: 1 or 2. The disease or condition may be related to or associated with serotonergic cells in the brain. The disease or condition may be chosen from: depression, bipolar disorder, anxiety, Parkinson's Disease, Huntington's Disease, Alzheimer's Disease, cognitive disorders, dementia, mood disorders, and schizophrenia.

The inventors herein further describe the present invention by way of the following non-limiting examples:

### WORKING EXAMPLES

General Methods

Expression Vector

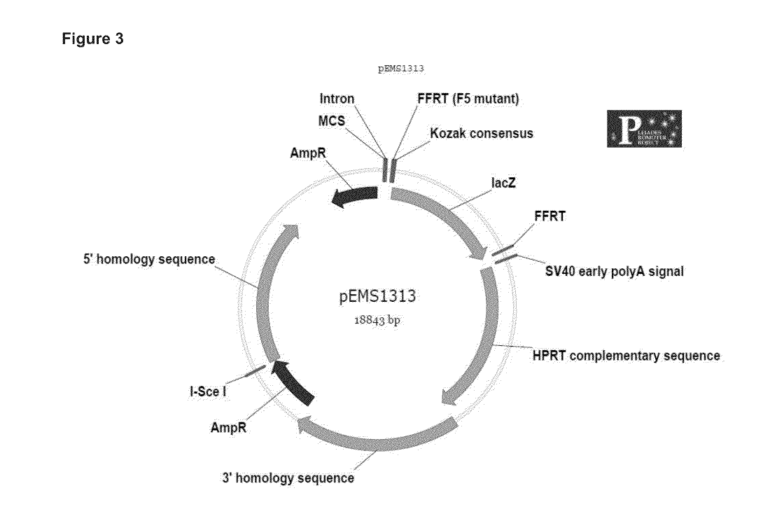

The nucleic acid fragment corresponding to SEQ ID NO: 1 or 2 was inserted into the multiple cloning site of pEMS1313 (driving the lacZ reporter, see FIG. 3) to produce the expression vectors (called Ple67 or Ple 313, respectively) used in the experiments.

Derivation of mEMS1202 Embryonic Stem Cells

Blastocysts were obtained from natural mating of B6-Hprtb-m3 homozygous females to 129-ROSA26 heterozygous males at 3.5 dpc. Blastocysts were flushed from uterine horns as per (Hogan, Beddington et al. 1994), cultured in EmbryoMax® KSOM with 1/2 Amino Acids, Glucose and Phenol Red (Cat #MR-121, Millipore/Chermicon, Temecula, Calif.) for 3-5 h, and then transferred onto mitomycin C (mitC; Cat #M4287, Sigma, Oakville, ON) mitotically inactivated B6-Hprtb-m3, B6129F1, or 129 mouse embryonic feeders (MEFs) derived from 13.5-day post-coital embryos (Ponchio, Duma et al. 2000) in 96-well plates containing KSR-ESC (Knockout™ D-MEM, Cat #10829-018, Invitrogen, Burlington, ON) with 2 mM L-glutamine (Cat #25030-081, Invitrogen, Burlington, ON), 0.1 mM MEM nonessential amino acid solution (Cat #11140-050, Invitrogen, Burlington, ON) and 16% Knockout™ Serum Replacement (Cat #10828-028, Invitrogen, Burlington, ON)) media (MEF media was replaced 3-5 hour prior to transfer). Blastocysts were cultured as per (Cheng, Dutra et al. 2004) with the following modifications: Cells were cultured for 7-9 days in KSR-ESC with minimal disturbance (checked on day 2 to determine if the blastocysts had ‘hatched’ out of the zona pellucida) and no media changes. Blastocysts which hatched and had a well-developed ICM (inner cell mass) were treated with 20 μl 0.25% trypsin-EDTA (Invitrogen, Burlington, ON) for 5 min at 37° C., triturated with a 200 μl Pipetman, inactivated with 30 μl 0.5 mg/ml soybean trypsin inhibitor (Invitrogen, Burlington, ON), and brought up to 200 μl with KSR-ESC, then transferred individually to a 24-well MEF plate containing 1800 μl KSR-ESC, for a total volume of 2 ml. Beginning 4 days later, KSR-ESC media was replaced with FBS-ESC media (DMEM (Cat #11960-069, Invitrogen, Burlington, ON) with 2 mM L-glutamine (Invitrogen, Burlington, ON), 0.1 mM MEM nonessential amino acid solution (Invitrogen, Burlington, ON), 16% ES Cell Qualified fetal bovine serum (FBS, Invitrogen, Burlington, ON), 1000 U ESGRO-LIF (Millipore, ESG1107) and 0.01% β-mercaptoethanol (Sigma, Oakville, ON)) in 25%, 50%, 75% proportions (respectively) to adapt the cells to FBS-containing media. On day 7 the cells were trypsinized to one well of a 24 well plate containing 1 ml of 100% FBS-ESC media, with daily media replacement. Once confluent, wells containing ESC colonies were expanded 3×24 wells (with MEFs), then passaged to 3×24 (with MEFs) and 3×12 well (plastic—no MEFs) for DNA analysis. Once confluent, the 3×24 wells were combined, aliquoted (3 vials), and frozen in ESC-freeze media (50% FBS, 40% FBS-ESC media, 10% DMSO (Sigma, Oakville, ON), and the 3×12 well treated with lysis buffer (Fisher Scientific, Ottawa, ON), mixed and aliquoted. Cultures were genotyped for X & Y chromosomes (Clapcote and Roder 2005), Gt(ROSA)26Sortm1Sor and WT alleles and Hprtb-m3 and WT alleles. B6129F1-Gt(ROSA)26Sortm1Sor/+, Hprtb-m3/Y (mEMS1204 series) and B6129F1-Gt(ROSA)26Sortm1Sor+/+, Hprtb-m3/Y (mEMS1202 series) cell lines were identified.

Knock-In at the Hprt Locus

The expression vector plasmid DNA was purified with Qiagen Maxi Kit (Qiagen, Mississauga, ON), resuspended in 10:1 Tris-EDTA (TE, pH7.0) buffer, and linearized with I-SceI (New England Biolabs, Pickering, ON). Linearized plasmid DNA was resuspended in 85 μl of TE (10:0.1) to a final concentration of 187.5 ng/μl. Ple155 was targeted in our in-house derived mEMS1202 cell line. ESCs were grown to confluence on 4-6 T75 flasks of mitC treated Hprtb-m3 mouse embryonic feeders (MEFs) in FBS-ESC media. ESCs (1.7-2.5×107) in 720 μl 1× PBS were added to the linearized DNA and electroporated in a 4 mm electroporation cuvette (Bio-Rad Genepulser, Mississauga, ON), at 240 V, 50 μF, 6-10 msec pulse, immediately resuspended in a total volume of 5 ml of FBS-ESC media and plated onto 5×100 mm dishes of mitC B6129F1 MEFs in a total volume of 12 ml per 100 mm dish. 24-36 h post-electroporation, correctly targeted homologous recombinants were selected for using HAT media (FBS-ESC media containing 1× HAT ((0.1 mM sodium hypoxanthine, 0.4 mM aminopterin, 0.16 mM thymidine), Cat #21060-017, Invitrogen, Burlington, ON). HAT media was changed every day for the first 3 days, and then every 3rd day thereafter, for up to 10 days. Individual colonies were counted and, typically, no more than 2 isolated colonies were picked per 100 mm dish to optimize for independent homologous recombination events. These colonies were expanded under standard protocols for verification of the desired recombination event.

Derivation of Knock-In Mice

Chimeric mice from targeted ESCs were generated by microinjection (Hogan, Beddington et al. 1994) into E3.5 blastocysts followed by implantation into the uterine horns of 2.5 day pseudopregnant ICR females. Chimeras were identified and coat color chimerism determined as outlined below.

Male chimeras derived from the E14TG2a cell lines were mated with B6 or B6-Alb females, and germline transmission was identified in the former case by the transmission of the dominant Aw (white bellied agouti) allele, making the progeny appear brown with a cream belly, or in the latter case by the combination of Aw and Tyrc-ch (chinchilla), making the progeny appear golden. Non-germline progeny from the cross to B6 were homozygous for the recessive a (nonagouti) allele and appeared black, whereas non-germline progeny from the cross to B6-Alb were homozygous for the recessive Tyrc-2J (albino 2 Jackson) allele and appeared white.

Male chimeras derived from the cell lines were mated with B6-Alb females, and germline transmission identified by the presence of the dominant Tyr+ (tyrosinase; wild type) and the Aw (white bellied agouti) or a (nonagouti) alleles making the progeny appear brown with a cream belly or black, respectively. Non-germline progeny were homozygous for the recessive Tyrc-2J (albino 2 Jackson) allele and appeared white. All germline female offspring carry the knock-in X Chromosome and were mated with B6 males.

N2 offspring were analyzed for the presence of the KI allele by PCR.

Reporter Gene Detection

Adult male hemizygous MiniPromoter and age matched control mice were perfused with 4% paraformaldehyde (PFA) as previously described (Young, Berry et al. 2002). Whole brains and eyes were dissected out and post-perfusion immersion fixed with PFA for 2 hours at 4° C. The brains were sectioned using a coronal or sagittal brain mold (Electron Microscopy Sciences) at 1 mm and sections were placed in 12-well tissue culture plates. One whole eye and one half-cut eye, using a razorblade, was also placed in the plate. LacZ expression was detected by using 5-bromo-4-chloro-3-indolyl-β-D-galactopyranoside (X-Gal) as the substrate. The X-Gal staining solution contained the following: 1.0 mg/ml X-Gal, 2 mM potassium ferricyanide, 2 mM potassium ferrocyanide, and 40 mM MgCl2 in PBS. In brief, brain sections were rinsed with phosphate buffered saline (PBS), then incubated with X-Gal (Boeringer Mannheim, Indianapolis, Ind.) at 37° C., usually overnight. After staining the tissue was rinsed with PBS and moved into PBS containing 0.02% azide for storage. Eyes were further processed by post-fixing with 4% PFA for 2 hours at room temperature. After fixation, eyes were rinsed with PBS and cryoprotected in 25% sucrose-PBS at 4° C. overnight. Eyes were removed from the solution and blotted with a KimWipe before embedment in Optimal Cutting Temperature (OCT) alongside positive and negative controls. 12 μm sections were taken using a Microm HM 550 cryostat and directly mounted onto SuperFrost Plus microscope slides. Bright field images were taken on a Leica MZ125 dissecting microscope and photographed using an Olympus Coolsnap cf color camera with the ImagePro software package.

### Example 1

### Expression of Reporter in Brain by Ple67

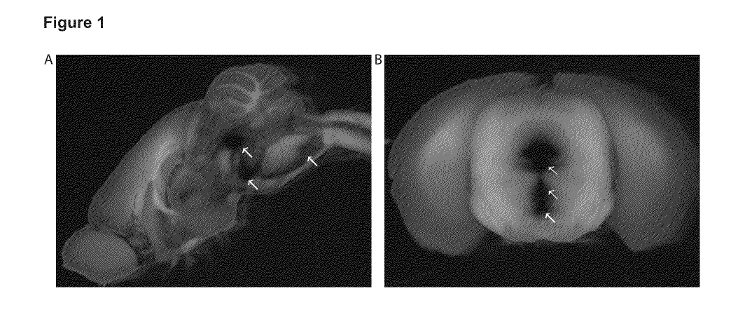

The Ple67 expression vector was introduced into mouse embryonic stem cells (ESCs) at the HPRT locus. The ESCs were used to generate genetically modified mice containing FEV. Immunohistochemical and immunofluorescence analysis of mouse brain tissue slices revealed lacZ reporter expression in the brain (see FIG. 1). Expression can be further characterized as follows. General: Overall labels most areas caudal of the nucleus accumbens and striatum with a sharp diagonal boundary. Expression throughout all Raphe Nuclei is evident. The labeling is consistent with that of serotonergic neurons throughout the mouse brain. Cortex: Mainly upper part of layer V (boundary between II/III and V). Most medially, layer II/III. Pallidum: Medial septal complex. Thalamus: Interoanteromedial nucleus. Strong in medial habenula. Hippocampus: Diffuse labeling. CA1 stratum oriens. Midbrain: Ventral tegmental area. Periaqueductal grey. Parapyramidal nucleus. Linear nucleus raphe. Dorsal raphe nucleus; superior central nucleus raphe. Hindbrain: Strong in pontine grey and superior central nucleus raphe (Pons). Nucleus raphe magnus, pallidus and obscurus (Medulla). Cerebellum: Purkinje cells clearly labeled, mostly negative otherwise.

### Example 2

### Expression of Reporter in Eye by Ple67

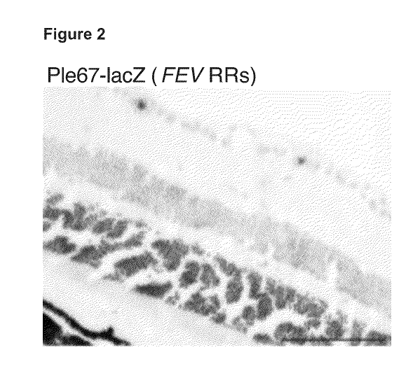

The Ple67 expression vecto was introduced into mouse embryonic stem cells (ESCs) at the HPRT locus. The ESCs were used to generate genetically modified mice containing FEV. Immunohistochemical and immunofluorescence analysis of mouse eye tissue slices revealed lacZ reporter expression in the eye (see FIG. 2). The Ple67 MiniPromoter, based on FEV putative regulatory regions, expresses in the retinal ganglion cell layer (GCL), with rare cells in the innermost aspect of the inner nuclear layer (INL) also positive. Staining is indicative of amacrine and/or retinal ganglion cells.

### Example 3

### Expression of Reporter by Ple313

Using the above methods, the Ple313 FEV DNA expression vector is introduced into mouse embryonic stem cells (ESCs) at the HPRT locus. The ESCs are used to generate genetically modified mice containing FEV mini-promoters. Immunohistochemical and immunofluorescence analysis of mouse brain and eye tissue slices are assessed for expression in certain cells of the eye, including retinal ganglion cells or retinal amacrine cells, and in the brain, including serotonergic cells. Evidence of expression in such cells in the mouse eye is determined by X-gal staining.

What is claimed is:

1. An isolated polynucleotide comprising a FEV mini-promoter.

2. The polynucleotide of claim 1 wherein the FEV mini-promoter comprises a FEV basal promoter with substantial similarity to SEQ ID NO: 1 or 2.

3. The isolated polynucleotide of claim 1-2, operably linked to an expressible sequence.

4. A vector comprising the isolated polynucleotide of claim 1-3.

5. A cell comprising the vector of claim 4.

6. The cell of claim 5, wherein the vector is stably integrated into the genome of the cell.

7. The cell of claim 5 or claim 6, wherein the cell is a stem cell or a retinal cell or a brain cell.

8. A method of expressing a sequence of interest, the method comprising operably linking the sequence of interest to the polynucleotide of claim 1-3; and introducing into a cell permissive for expression from the FEV mini-promoter.

9. A method of treatment of a subject having a disease or condition of the central nervous system, the method comprising administering to the subject a therapeutically effective dose of a composition comprising the polynucleotide or cell of claim 1-7.

10. A method of treatment of a subject having a disease or condition of the eye, the method comprising administering to the subject a therapeutically effective dose of a composition comprising the polynucleotide or cell of claim 1-7.


SEQUENCE LISTING (2 sequences)

SEQ ID NO: 1 - DNA, 2202 bp, H. sapiens
ggagaagatcaagactcaggtgggtggagatgactgccccccatgcacacatttcctggtgctccaccccctcagcaaatgacttctgggtaagactgagggataaagcctatactgttgatccttgcccagctcacagctgggatatttgggtagacctcactaggaccctctcctttacaccttagcctcccactgagagcattaagtgggcgaattttgatgcctcccagctttcccagctcttacctcctgcccctggctattttctgttcttacctcctgctccttgttttctgtccatagtgggtgggggaagcagaaagaggaatgagaaggagagggattgggatcttgaccccaacttcaaggccctcaccttctgtggagccctgagtttggtccacagggaacattccaacctctagtcattgctcccaccagattccaggtccacatcctgaaggcttcatccctgtcccactaggatcctcccaggccctgggatccctctttttccctccacactctgcctcccactcttctccctggctcctttgcaccctccacgagcaaagatgaacactccagaagttgcagcaaatgagggtgaacatgggccctgtagaaccccagaagtaggagtttccagccatcctcaatcaggcatcccaggaccaggcacttagcaggagacctgcccattcccaggcctcctccacccctgggctcaagcagtgagagaggactttgagaatgccagttttgacagtacccgttccatactgcaaggctgcagaaggtgggcttctgggctggagcctcatcttcagccagcatcctccttagcctgcctctccagagccacgaagccccataggtacccagagagactccaggccacttgggaaccacatcaacatctttgcatgagttagaaataggaatccttttcttggagtggattccagccttgtacagtgcacaaccctgtcttaacccctccacctctccagcctcgttcagggatagccagaatgggagtagtggtgagggtgggtggcagactgggaggacaggggatggggaaggatgggagcagaatagttgtacagggaagtagcagggggctcaggaacccatcagggaaagccaggacagattctcaacatctttagaggccttcagcagtccaagccactactaatttatggggactgaatgaatgagaaaaggaggggagggagaaggatgagagggagagagaaggctggaggggagatgagagtcggagagaaaagcctaggaggtaaagagagacctagaagggggcggggaggacagaagagggggccacccatcataagttgccaggagagatggcccagcaccaggcctgaggcttgggggccccagaaaggcaggaaacgggccaaagcaagaaggagacggggaaggggttcgggtctctagggaagagcagagagtgggagaagaggaggagggagcggggatgcgggcagagataaagggagcaacggctgcagggccgggcgcgcagcgacaggcgagagggagggaagcggaaatttaaaagtgaagatgcagataacgcagcctggagacgggaacccgggtggggatagagggagggagggggaggagagtgagcggcgagtggagcgggagccgactggaagaagggctctagggagggggctgtggctgctggggtccgaggtggggccgggtacaccagccccatcactgtttgcagagagtcagggaggcggaaaagacacgcgctctaggctcccatcagggcacatggcccggacccatcccccgcgcgtctccccggctgcggggcgcggggggctgccgggtgcgcttggctgtggcgcggcgcgttggagactttattgcgatgggacgataagaggggcgggggcggggtcctgggggccgaggcggcagcgctttaattaaaacggaaattgcggccccgggccgcgcgggggccggagggttccaagcggccccttagctggaagcgtttctccaggacccccccgcaacccccgccacgcccgggctgccccctcccgccaggccctgccggacccggcgccgtcttctcctccttgtcacccgcggtcgcttcgggcggggatcggtgccaccgagcgcaaagcctgcctc

SEQ ID NO: 2 - DNA, 1650 bp, H. sapiens
ctcctttgcaccctccacgagcaaagatgaacactccagaagttgcagcaaatgagggtgaacatgggccctgtagaaccccagaagtaggagtttccagccatcctcaatcaggcatcccaggaccaggcacttagcaggagacctgcccattcccaggcctcctccacccctgggctcaagcagtgagagaggactttgagaatgccagttttgacagtacccgttccatactgcaaggctgcagaaggtgggcttctgggctggagcctcatcttcagccagcatcctccttagcctgcctctccagagccacgaagccccataggtacccagagagactccaggccacttgggaaccacatcaacatctttgcatgagttagaaataggaatccttttcttggagtggattccagccttgtacagtgcacaaccctgtcttaacccctccacctctccagcctcgttcagggatagccagaatgggagtagtggtgagggtgggtggcagactgggaggacaggggatggggaaggatgggagcagaatagttgtacagggaagtagcagggggctcaggaacccatcagggaaagccaggacagattctcaacatctttagaggccttcagcagtccaagccactactaatttatggggactgaatgaatgagaaaaggaggggagggagaaggatgagagggagagagaaggctggaggggagatgagagtcggagagaaaagcctaggaggtaaagagagacctagaagggggcggggaggacagaagagggggccacccatcataagttgccaggagagatggcccagcaccaggcctgaggcttgggggccccagaaaggcaggaaacgggccaaagcaagaaggagacggggaaggggttcgggtctctagggaagagcagagagtgggagaagaggaggagggagcggggatgcgggcagagataaagggagcaacggctgcagggccgggcgcgcagcgacaggcgagagggagggaagcggaaatttaaaagtgaagatgcagataacgcagcctggagacgggaacccgggtggggatagagggagggagggggaggagagtgagcggcgagtggagcgggagccgactggaagaagggctctagggagggggctgtggctgctggggtccgaggtggggccgggtacaccagccccatcactgtttgcagagagtcagggaggcggaaaagacacgcgctctaggctcccatcagggcacatggcccggacccatcccccgcgcgtctccccggctgcggggcgcggggggctgccgggtgcgcttggctgtggcgcggcgcgttggagactttattgcgatgggacgataagaggggcgggggcggggtcctgggggccgaggcggcagcgctttaattaaaacggaaattgcggccccgggccgcgcgggggccggagggttccaagcggccccttagctggaagcgtttctccaggacccccccgcaacccccgccacgcccgggctgccccctcccgccaggccctgccggacccggcgccgtcttctcctccttgtcacccgcggtcgcttcgggcggggatcggtgccaccgagcgcaaagcctgcctc